(19) United States
(12) Patent Application Publication
(10) Pub. No.: US 2021/0128763 A1
(43) Pub. Date: May 6, 2021

(54) PERFORMANCE MONITORING SYSTEM AND METHOD FOR AN ADVANCED OXIDATION PROCESS (AOP) WATER SANITIZER

(71) Applicant: CUSTOM MOLDED PRODUCTS, LLC, Newnan, GA (US)

(72) Inventors: Karthik Hosavaranchi PUTTARAJU, Peachtree City, GA (US); Zachary T. VOGTNER, Fayetteville, GA (US); Victor L. WALKER, Temecula, CA (US)

(73) Assignee: CUSTOM MOLDED PRODUCTS, LLC, Newnan, GA (US)

(21) Appl. No.: 16/673,001

(22) Filed: Nov. 4, 2019

(51) Int. Cl. A61L 2/10; A61L 2/18

(52) U.S. Cl. CPC A61L 2/10; A61L 2202/11; A61L 2/183

(57) ABSTRACT

A system for monitoring performance of a water sanitation device includes a housing having a water flow path, a power source, an ozone generating element configured to provide ozone to the water flow path, and an ultraviolet (UV) light generating element configured to expose the water in the flow path to UV light, a first monitoring circuit configured to monitor at least one operational aspect of the ultraviolet (UV) light generating element, a second monitoring circuit configured to monitor at least one operational aspect of the ozone generating element, a control circuit configured to receive an output of the first monitoring circuit and an output of the second monitoring circuit, and a display element configured to provide an indication of a status of at least one of the power source, the ultraviolet (UV) light generating element and the ozone generating element.

### FIELD

The technology described herein relates to water sanitation treatment devices and more particularly to a system and method for monitoring the status and performance of a water sanitation treatment device.

### BACKGROUND

An advanced oxidation process (AOP) water treatment and sanitation system operates by exposing ozone in the water to germicidal UV light (UV-C) rays which produces hydroxyl radicals. Ozone may be generated by an ozone-producing element, sometimes referred to as an ozone generator cell or an ozone generating cell. When germicidal UV light and ozone react, the result is the production of hydroxyl radicals. Hydroxyl radicals have the highest oxidation potential of any residential application water sanitizer. The hydroxyl radicals produced by AOP are generally more powerful than chlorine and other known sanitizers, and generally more powerful than ozone alone. In AOP systems, the highly unstable hydroxyl radicals react with dissolved waterborne contaminants in a series of strong oxidation reactions to treat the water.

An AOP system relies on the generation of ozone and the exposure of the ozone to germicidal UV light. Over time, the performance of ozone generating cells and the UV lamps will diminish and will no longer be effective at treating the water. The UV lamps and ozone cells should be periodically replaced to maintain their effectiveness.

One problem with monitoring the performance of the ozone generating cells and the UV lamps is that they do not provide sufficient feedback to indicate that maintenance or replacement is due or past due. Performance of these components is difficult to judge visually, and the performance cannot be readily measured in the field. Therefore, it would be advantageous to provide a system and method to monitor these components and provide visual feedback to the user as to the useful service life and the effectiveness of the components.

Moreover, because AOP is relatively new to the recreational water industry (swimming pools, hot tubs, water parks, splash pads, etc.), many industry professionals are not familiar with them, and as a result, the likelihood of installation errors may increase. Current AOP products do not provide sufficient feedback to indicate if there are installation issues, such as, for example, a wiring or a power issue. Therefore, it may also be advantageous to provide visual feedback of these installation issues to the user.

### SUMMARY

Various implementations of systems, methods and devices within the scope of the appended claims each have several aspects, no single one of which is solely responsible for the desirable attributes described herein. Without limiting the scope of the appended claims, some prominent features are described herein.

Details of one or more implementations of the subject matter described in this specification are set forth in the accompanying drawings and the description below. Other features, aspects, and advantages will become apparent from the description, the drawings, and the claims. Note that the relative dimensions of the following figures may not be drawn to scale.

One aspect of the disclosure provides a system for monitoring performance of a water sanitation device includes a housing having a water flow path, a power source, an ozone generating element configured to provide ozone to the water flow path, and an ultraviolet (UV) light generating element configured to expose the water in the flow path to UV light, a first monitoring circuit configured to monitor at least one operational aspect of the ultraviolet (UV) light generating element, a second monitoring circuit configured to monitor at least one operational aspect of the ozone generating element, a control circuit configured to receive an output of the first monitoring circuit and an output of the second monitoring circuit, and a display element configured to provide an indication of a status of at least one of the power source, the ultraviolet (UV) light generating element and the ozone generating element.

Another aspect of the disclosure provides a method for monitoring performance of a water sanitation device including providing sensor data relating to an operational aspect of one or more of an ozone generating element and an ultraviolet (UV) light generating element to a controller, determining whether the sensor data indicates that a first threshold has been met, if the first threshold has been met, causing an illumination of a first indicator signifying that the first threshold has been met, determining whether the sensor data indicates that a second threshold has been met, if the second threshold has been met, causing an illumination of a second indicator signifying that the second threshold has been met, determining whether the sensor data indicates that a condition that caused the first threshold and the second threshold to be met has been removed, and if the sensor data indicates that the condition has not been removed, causing an illumination of a third indicator signifying that the condition has not been removed.

Another aspect of the disclosure provides a method for monitoring performance of a water sanitation device including determining whether a fault in one or more of an incoming power level, an ultraviolet (UV) light generating element and an ozone generator cell exists, if a fault exists, causing an illumination of a first indicator signifying that the fault exists, and if the fault is remedied, ceasing the illumination of the first indicator.

### BRIEF DESCRIPTION OF THE DRAWINGS

In the figures, like reference numerals refer to like parts throughout the various views unless otherwise indicated. For reference numerals with letter character designations such as “102a” or “102b”, the letter character designations may differentiate two like parts or elements present in the same figure. Letter character designations for reference numerals may be omitted when it is intended that a reference numeral encompass all parts having the same reference numeral in all figures.

FIG. 1 is a schematic view of an advanced oxidation process (AOP) water treatment and sanitation system.

FIG. 2 is a schematic view of the advanced oxidation process (AOP) water treatment and sanitation system of FIG. 1.

FIG. 3A is a schematic view of the advanced oxidation process (AOP) water treatment and sanitation system of FIG. 1.

FIG. 3B is a schematic view of an indicator system of the advanced oxidation process (AOP) water treatment and sanitation system of FIG. 1.

FIG. 3C is a schematic view of a rear portion of the indicator system of the advanced oxidation process (AOP) water treatment and sanitation system of FIG. 1.

FIG. 4 is a block diagram showing an exemplary embodiment of an electrical circuit associated with the advanced oxidation process (AOP) water treatment and sanitation system of FIG. 1.

FIG. 5 is a schematic diagram showing an exemplary embodiment of a current sensing circuit of the AOP system of FIG. 1.

FIG. 6 is a schematic diagram showing an exemplary embodiment of a current sensing circuit of the AOP system of FIG. 1.

FIG. 7 is a block diagram showing an exemplary embodiment of an electrical circuit associated with the advanced oxidation process (AOP) water treatment and sanitation system of FIG. 1.

FIG. 8 is a block diagram showing an exemplary embodiment of an electrical circuit associated with the advanced oxidation process (AOP) water treatment and sanitation system of FIG. 1.

FIG. 9 is a block diagram showing an exemplary embodiment of an electrical circuit associated with the advanced oxidation process (AOP) water treatment and sanitation system of FIG. 1.

FIG. 10 is a flow chart describing an example of the operation of an AOP system.

FIG. 11 is a flow chart describing an example of the operation of an AOP system.

### DETAILED DESCRIPTION

The following description, and the figures to which it refers, are provided for the purpose of describing examples and specific embodiments of the invention only and are not intended to exhaustively describe all possible examples and embodiments of the invention.

The word “exemplary” is used herein to mean “serving as an example, instance, or illustration.” Any aspect described herein as “exemplary” is not necessarily to be construed as preferred or advantageous over other aspects.

An AOP sanitizer generally performs at its peak when maintained at regular intervals for both the ozone generating cells and the UV lamps. Ozone generating cells should be cleaned or replaced periodically. The UV lamp (or lamps) should be replaced periodically and the quartz tube (or tubes) in which they are mounted should be cleaned periodically to ensure that sufficient UV light is transmitted through the tubes and into the water. Maintenance intervals are dependent upon the component manufacturer's ratings for effective service life. If the components are used beyond the effective service life, the performance of that component diminishes and the AOP sanitizer no longer effectively sanitizes the water. If used for a swimming pool, for example, there is no indication to the pool owner that the components are beyond their useful life, and the pool owner may be unknowingly operating an unsafe pool.

In the case of an ozone generating cell, there is no practical way to measure the amount of ozone produced, and the hydroxyl radical output resulting from the exposure of the ozone to UV light cannot be judged visually. Measuring the performance of the UV lamps and ozone generating cells individually requires expensive equipment, and cannot be done effectively in the field after installation. Therefore, indication on the water sanitizing product containing the ozone generating cells and the UV lamps is the only way to determine whether the water sanitizing product is effectively sanitizing the water.

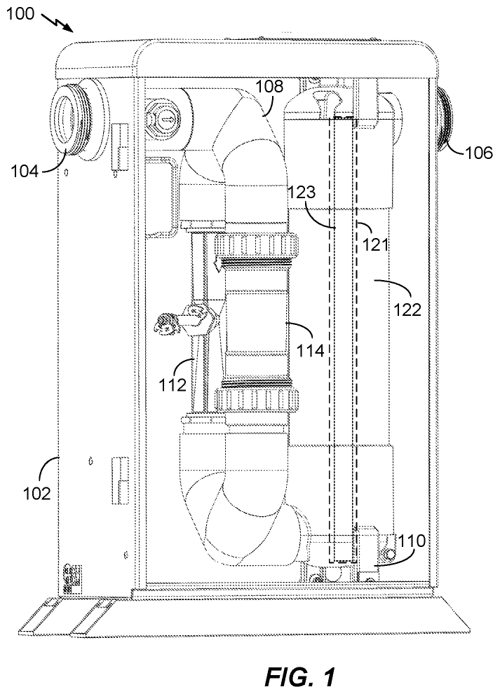

FIG. 1 is a schematic view of an advanced oxidation process (AOP) water treatment and sanitation system 100, hereafter referred to as the AOP system 100. The AOP system 100 comprises a housing 102, a flow inlet 104, a flow outlet 106 and a drain port 110. The flow inlet 104 couples to a flow path 108. In an exemplary embodiment, the flow path 108 may comprise a first flow path 112 and a second flow path 114. However, a single flow path may also be implemented. The flow path 108 includes a UV light chamber 122. The UV chamber 122 may comprise a quartz tube 121 within which a UV lamp 123 may be located. Water may enter the flow inlet 104, travel through the flow path 108 and the UV light chamber 122, and exit through the flow outlet 106. In an exemplary embodiment, the water may flow through one or more of the first flow path 112 and the second flow path 114.

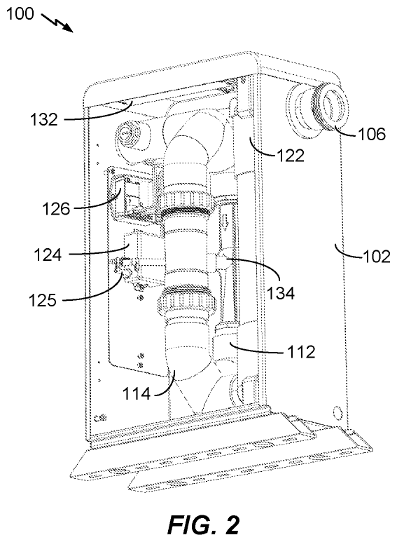

FIG. 2 is a schematic view of the advanced oxidation process (AOP) water treatment and sanitation system 100 of FIG. 1. In an exemplary embodiment, the AOP system 100 includes the UV light chamber 122 having the UV lamp 123 (FIG. 1) configured to produce UV light (not shown in FIG. 2), an ozone generator cell 124, and control circuit 126. A UV ballast 132 may be electrically coupled to an electrical power source and may be electrically coupled to the UV lamp 123 (FIG. 1) inside the UV chamber 122. The UV chamber 122 may comprise the UV light-transparent quartz tube 121 (FIG. 1) in which the UV lamp 123 (FIG. 1) is located. The ozone generator cell 124 may have an output port 125, which may be fluidically coupled to the first flow path 112 or to the second flow path 114 to introduce ozone (03) into the flow of water traveling through the flow path 108, and in an exemplary embodiment, through the first flow path 112. In an exemplary embodiment, the ozone generator cell 124 may be fluidically coupled to the first flow path 112, which may comprise a venturi section (referred to as a venturi ozone injector) 134 to facilitate the introduction of ozone produced by the ozone generator cell 124 into the flow of water passing through the first flow path 112. In an exemplary embodiment, at least some portions of the first flow path 112 and second flow path 114 may be transparent to facilitate observation of water flowing through the flow path 108. In an exemplary embodiment, UV light generated by the UV lamp 123 (not shown in FIG. 2) inside the UV chamber 122 exposes the ozone-rich water passing through the UV chamber 122 to UV light so that hydroxyl radicals may be produced. In this manner, the AOP system 100 may be configured to treat water passing through the flow path 108 with hydroxyl radicals, ozone and UV light.

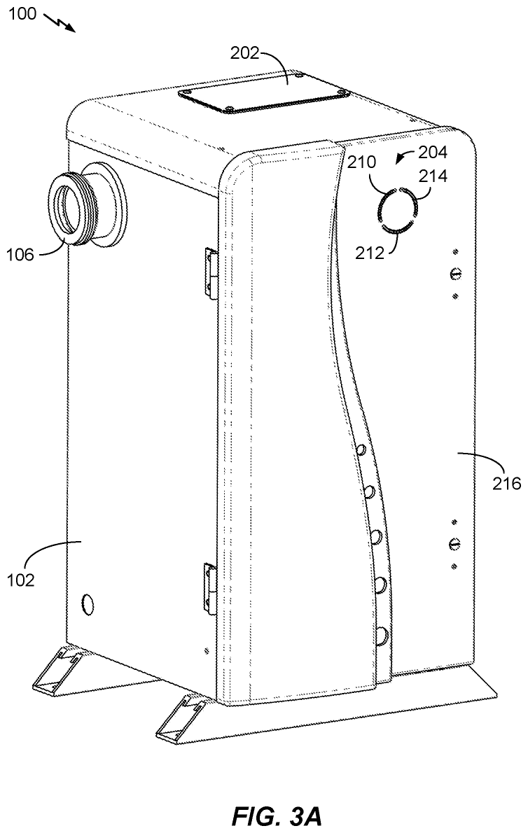

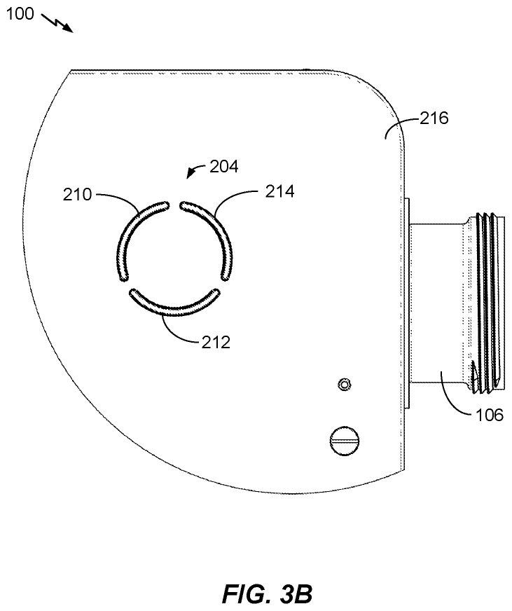

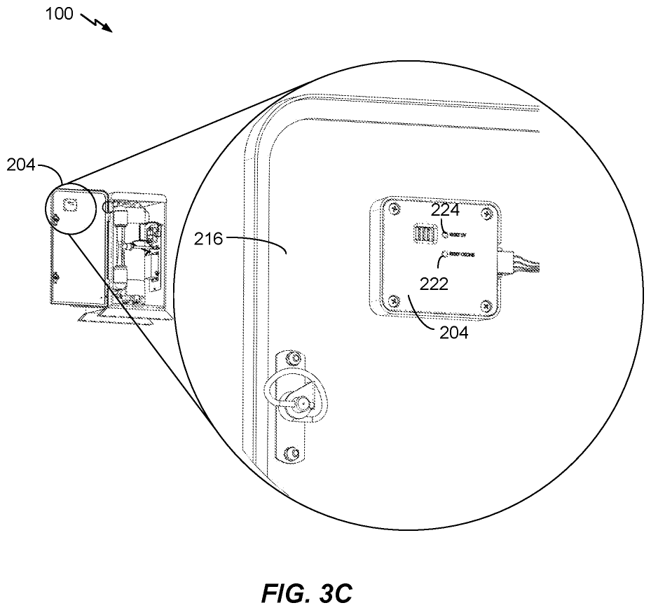

FIGS. 3A, 3B and 3C are schematic views of the advanced oxidation process (AOP) water treatment and sanitation system 100 of FIG. 1. The view of the AOP system 100 in FIG. 3A also shows an access hatch 202 configured to allow access to the UV lamp (not shown) in the UV chamber 122 (not shown in FIG. 3A). The AOP system 100 also comprises an indicator system 204 located on a door 216. In an exemplary embodiment, the indicator system 204 may comprise one or more light emitting diodes (LEDs) configured to illuminate based on the operating condition of one or more systems of the AOP system 100. As shown in FIG. 3B, in an exemplary embodiment, the indicator system 204 may comprise an LED 210 associated with the power status of the AOP system 100, an LED 214 associated with the ozone generator cell of the AOP system 100 and an LED 212 associated with the UV lamp of the AOP system 100. In an exemplary embodiment, each LED may be configured to illuminate in one or more colors to indicate the operating status, maintenance status, or other status, of the above-mentioned systems of the AOP system 100.

A rear side of the indicator system 204, as shown in FIG. 3C, may comprise an ozone indicator reset switch 222 configured to reset the LED 214 and may comprise a UV indicator reset switch 224 configured to reset the LED 212.

In an exemplary embodiment, the indicator system 204 may comprise other types of indicators, such as, for example only, a display such as a liquid crystal display (LCD) configured to show the operational status of the above-described systems of the AOP system 100. In alternative exemplary embodiments, the indicator system 204 may comprise one or more of audible indicators, tactile indicators, or other indicators configured to convey the operational status of the above-described systems of the AOP system 100.

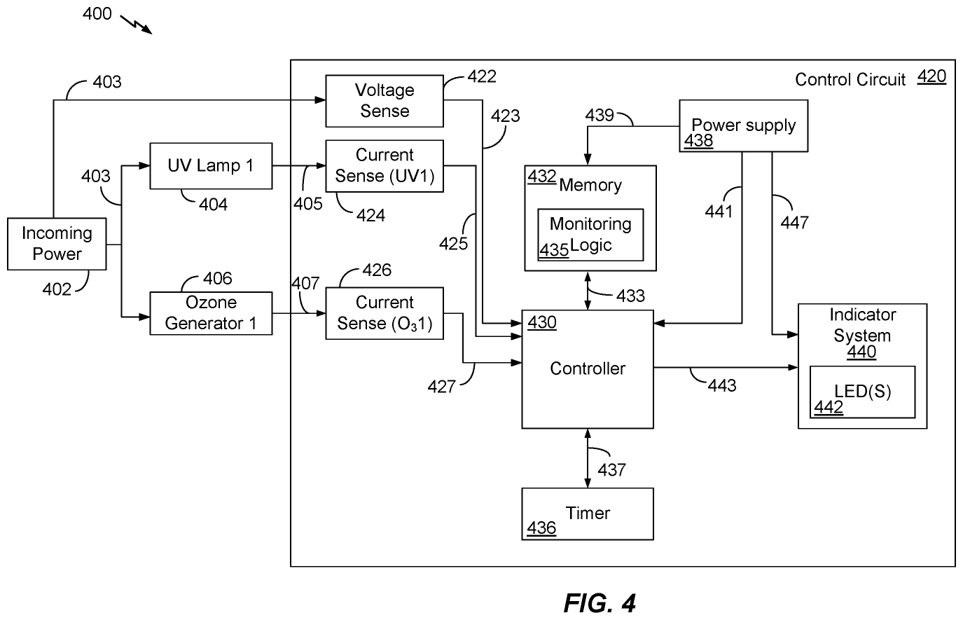

FIG. 4 is a block diagram showing an electrical subsystem 400 associated with the advanced oxidation process (AOP) water treatment and sanitation system of FIG. 1. In an exemplary embodiment, the electrical subsystem 400 may comprise one or more components or elements that may be located in the housing 102 of the AOP system 100. In an exemplary embodiment, the electrical subsystem 400 may comprise an incoming power distribution element 402 configured to provide incoming electrical power. In an exemplary embodiment, the incoming electrical power may be, for example, nominal 110 volts alternating current (VAC), 240 VAC, or another incoming voltage level. In an exemplary embodiment, the incoming power distribution element 402 may be configured to provide electrical power to a control circuit 420 and to one or more electrical components, such as, in this exemplary embodiment, to a UV light generating element 404 and to an ozone generator cell 406. In an exemplary embodiment, the UV light generating element 404 may be a UV lamp 123 (FIG. 1) contained in the UV chamber 122 (FIG. 2) and the ozone generator cell 406 may be an exemplary embodiment of an ozone generator cell 124 (FIG. 1). In an exemplary embodiment, the control circuit 420 may be an example of the control circuit 126 of FIG. 2.

In an exemplary embodiment, the control circuit 420 may comprise a voltage sense element 422, a first current sense element 424 and a second current sense element 426. In an exemplary embodiment, the first current sense element 424 and the second current sense element 426 may be referred to as first and second monitoring circuits, respectively. In an exemplary embodiment, the voltage sense element 422 may be configured to sense one or more operational aspects of the incoming power distribution element 402, such as the voltage output on connection 403 and provide a signal output on connection 423 to a controller 430, the signal being indicative of the voltage output on connection 403.

In an exemplary embodiment, the first current sense element 424 may be configured to receive a signal over connection 405 from the UV light generating element 404, and provide a signal over connection 425 to the controller 430 that is indicative of one or more operational aspects of the UV light generating element 404. For example, the signal on connection 425 provided by the first current sense element 424 may be indicative of an installation status of the UV light generating element 404. In another example, the signal on connection 425 provided by the first current sense element 424 may be indicative of whether the UV light generating element 404 is operating under normal operating conditions.

In an exemplary embodiment, the second current sense element 426 may be configured to receive a signal over connection 407 from the ozone generator cell 406, and provide a signal over connection 427 to the controller 430 that is indicative of one or more operational aspects of the ozone generator cell 406. For example, the signal on connection 427 provided by the second current sense element 426 may be indicative of installation status of the ozone generator cell 406. In another example, the signal on connection 427 provided by the second current sense element 426 may be indicative of whether the ozone generator cell 406 is operating under normal operating conditions.

In an exemplary embodiment, the control circuit 420 also comprises a controller 430 operatively coupled to a memory 432 over connection 433. A power supply 438 may be coupled to the controller 430 over connection 441, to the memory 432 over connection 439 and to an indicator system 440 over connection 447. The controller 430 may be coupled to a timer 436 over connection 437. The memory 432 may include monitoring logic 435 containing instructions, software, firmware, code or other logic for performing the functions described herein.

In an exemplary embodiment, the memory 432 may be a discrete element such as that shown in FIG. 4, or may be a distributed memory, or may be integrated with the controller 430 or with other elements in the control circuit 420. The memory 432 may comprise a random-access memory (RAM), a read-only memory (ROM), an electrically erasable programmable ROM (EEPROM), optical disk storage, magnetic disk storage, or other magnetic, optical, electronic, or other storage devices.

The controller 430 may be a microcontroller, a microprocessor, an application specific integrated circuit (ASIC), a field programmable gate array (FPGA), or any other processor or controller capable of executing the instructions in the memory 432 and in the monitoring logic 435.

Although shown as discrete elements, the controller 430, memory 432 and timer 436 may be implemented together in a single element. Further, the connections 433, 437 and 443 may be combined on a signal and/or logic bus. Similarly, although shown as a discrete element, an indicator system 440 may be incorporated or integrated with one or more elements on the control circuit 420.

The indicator system 440 may be coupled to the controller 430 over connection 443 and to the power supply 438 over connection 447. In an exemplary embodiment, the power supply 438 may be configured to provide an AC voltage or a DC voltage. In an exemplary embodiment, the power supply 438 may be part of or coupled to a solar power system.

In an exemplary embodiment, the indicator system 440 may be an example of the indicator system 204 of FIG. 3A, FIG. 3B and FIG. 3C. In an exemplary embodiment, the indicator system 440 may comprise one or more light emitting diodes (LEDs), configured to be illuminated by the controller 430 upon the occurrence of certain performance and/or maintenance events. In an exemplary embodiment, the indicator system 440 may comprise three LEDs comprising a power indicator LED, a UV light generating element LED and an ozone generator cell LED, each LED capable of illuminating in multiple colors, and in flashing or blinking patterns.

The controller 430 may be configured to receive the output of the voltage sense element 422, the first current sense element 424 and the second current sense element 426, and process those outputs to determine one or more operational aspects or operating conditions of the incoming power distribution element 402, the UV light generating element 404 and the ozone generator cell 406. For example, in an exemplary embodiment, the controller 430 may execute the monitoring logic 435 in the memory 432 and determine and store in the timer a total time of operation of one or more of the UV light generating element 404 and the ozone generator cell 406. The total time of operation may be determined by monitoring the total time that the output of one or more of the first current sense element 424 and the second current sense element 426 is maintained within a certain predefined range of current values. For example, if the current output of one or more of the first current sense element 424 and the second current sense element 426 remains within a predefined working current range, then the controller 430 will cause the timer 436 to run, and accumulate the total operating time of one or more of the UV light generating element 404 and the ozone generator cell 406. An example of a working current range for an ozone generator cell may be, for example, approximately 50 mA (milliamps) to approximately 500 mA. An example of a working current range for a UV light generating element may be, for example, approximately 250 mA to approximately 1.5 A. Other elements may have other operating current ranges, and the ones given here are for example purposes only. If the current range falls out of these exemplary ranges, then a fault may be registered and the time that the current falls out of these exemplary ranges would not be counted as operating time for that particular component. In an exemplary embodiment, the controller 430 may then compare the total operating time for one or more of the UV light generating element 404 and the ozone generator cell 406 against one or more predetermined time periods, and when the one or more predetermined time periods are met or exceeded, the controller 430 can cause one or more LEDs 442 in the indicator system 440 to illuminate based on the detected condition.

In another exemplary embodiment, as will be described further herein, if the operating current remains within the operating current range, but is close to an edge or limit of the operating current range, then the controller 430 may be configured to generate an alert or warning that may be communicated to a user indicating that the component that is close to the edge or limit of the operating range may be operating inefficiently, or operating not as efficiently as desired.

The timer 436 may be a discrete element configured to monitor and maintain the operational time, or operating time, or total operating time, of one or more of the UV light generating element 404 and the ozone generator cell 406.

An example of the LED indicator condition based on the operational status of the AOP system 100 is shown in Table 1.

TABLE 1
LED Indicator Reference

| | Power | UV | Ozone |
|---|---|---|---|
| Status | LED | LED | LED |
| Fully Operational - no faults or service due | Green | Purple | Blue |
| Fault with incoming power or other fault not | Red | Purple | Blue |
| related to UV or ozone cells | | | |
| Fault with UV unit | Red | Red | Blue |
| Fault with Ozone unit | Red | Purple | Red |
| After UV lamps have been energized for 16 | Green | Yellow | Blue |
| months (~11.5 khrs) and up to 18 months (~13k | | | |
| hrs) without service | | | |
| After UV lamps have been energized for 18 | Yellow | Blinking | Blue |
| months (~13 khrs) without service | | Red | |
| After ozone cell has been energized for 28 | Green | Purple | Yellow |
| months (~20k hrs) and up to 30 months (~21.5k | | | |
| hrs) without service | | | |
| After ozone cell has been energized for 30 | Yellow | Purple | Blinking |
| months (~21.5 khrs) without service | | | Red |
| UV Lamp not serviced within 18 months (~13k | Red | Blinking | Blinking |
| hrs) and Ozone not serviced within 30 months | | Red | Red |
| (~21.5k hrs) | | | |

An AOP sanitizer benefits from maintenance at regular intervals for both the ozone generating cells 406 and the UV light generating elements 404. Ozone generating cells should be cleaned or replaced periodically. UV lamps in the UV light generating elements 404 should be replaced periodically and the quartz tubes in which they are located should be cleaned periodically to ensure that ample UV light is transmitted through the tubes and into the water. Maintenance intervals are dependent upon the component manufacturer's ratings for effective service life. If the components are used beyond their effective service life, the performance of that component diminishes and the AOP sanitizer may no longer effectively sanitize the water. If there is no indication to the pool owner that the components are beyond their service life, they may be unknowingly operating an unsafe pool.

It is generally impractical for a user to measure the amount of hydroxyl radicals produced by an AOP system 100, and the hydroxyl radical output cannot be judged visually. Measuring the performance of the UV lamps and ozone generating cells individually requires expensive equipment, and cannot be done effectively in the field after installation. Therefore, an indication on the product is the only way the pool owner can tell if the product is effectively sanitizing the water.

In an exemplary embodiment, an ozone generating cell is certified to have an effective life of approximately 30 months continuous use, and the UV lamps are certified to approximately 18 months continuous use. In an exemplary embodiment, the AOP system 100 described herein monitors and stores the total time that each component is in operation, that is, the “operating” or “on” time of the UV light generating element 404 and of the ozone generating cell 406 is monitored and stored by the controller 430, timer 436, memory 432 and monitoring logic 435. If the UV lamp or the ozone generating cell is not energized and not consuming a predetermined amount of current, in this example, then the monitoring logic 435 may consider that time as “non-operating” time and the monitoring logic 435 would not count that “non-operating” time toward the total life of the UV lamp or of the ozone generating cell. When one or more of the UV light generating element 404 and the ozone generating cell 406 reaches within a predetermined time (for example, 2 months) of its service life, the monitoring logic 435 causes the controller 430 to cause the indicator system 440 to change the indicator LED from, for example green, to, for example, yellow. Subsequently, if the UV light generating element 404 or the ozone generating cell 406 reaches the end of, or goes beyond its service life, the monitoring logic 435 causes the controller 430 to cause the indicator system 440 to change the indicator LED from, for example yellow, to, for example, red, signifying the end of the service life of that component. If the UV light generating element 404 or the ozone generating cell 406 is not replaced, the monitoring logic 435 causes the controller 430 to cause the indicator system 440 to change the indicator LED from, for example red, to, for example, blinking red, signifying that the end of life has been reached and the component has not been replaced.

By utilizing separate indicators for ozone and UV the monitoring logic 435 can indicate maintenance for each component individually.

After maintenance is completed, the user actuates the appropriate reset button 222, 224 located on the back of the door 216 of the AOP system 100. In this embodiment, there are two reset buttons—one for resetting the ozone generator cell monitoring function (reset button 222) and one for resetting the UV light generating element monitoring function (reset button 224). In a system with two or more UV light generating elements or two or more ozone generator cells, it is possible to have multiple individual reset buttons to correspond with each of the two or more UV light generating elements or two or more ozone generator cells. Pressing the reset button resets the timer 436 that monitors operation time for each component. The reset can occur after the applicable button is held down for an extended duration so as to avoid resetting with accidental contact. In another exemplary embodiment, the reset buttons may be recessed or use a special tool to activate or actuate.

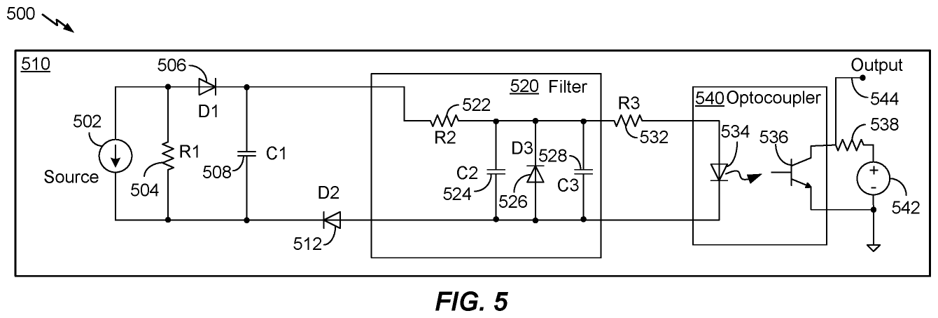

FIG. 5 is a schematic diagram showing an exemplary embodiment of a current sense circuit 500 of the AOP system of FIG. 1. The current sense circuit 500 is referred to as an “electrically isolated” or “isolated” circuit. In an exemplary embodiment, the current sense circuit 500 comprises a current source 502 and a resistor 504. In an exemplary embodiment, the current sense circuit 500 is configured to monitor the voltage drop across the resistor 504 caused by the current flowing through, or consumed by, the current source 502. In an exemplary embodiment, the current sense circuit 500 may be an example of the first current sense element 424 or the second current sense element 426 of FIG. 4.

The example current sense circuit 500 also includes a diode 506, a capacitor 508 and a diode 512. In a non-limiting example implementation, the diode 506 and the diode 512 may be implemented using Schottky diodes.

The example current sense circuit 500 also comprises a filter 520. In an exemplary embodiment, the filter 520 comprises a resistor 522, a capacitor 524, a diode 526 and a capacitor 528. In an example implementation, the diode 536 may be a Zener (or voltage regulator) diode. The output of the filter 520 is provided to the resistor 532. In the example implementation shown in FIG. 5, the current sense circuit 500 also comprises an optocoupler 540, which includes a light emitting diode 534 and an optical-to-electrical converter 536. The output of the optocoupler 540 is provided to the resistor 538 and a voltage source 542. In an exemplary embodiment, the output of the current sense circuit 500 may be taken over node 544. In alternative exemplary embodiments, the output of the current sense circuit 500 may be taken from the ground side of the voltage source 542, depending on implementation.

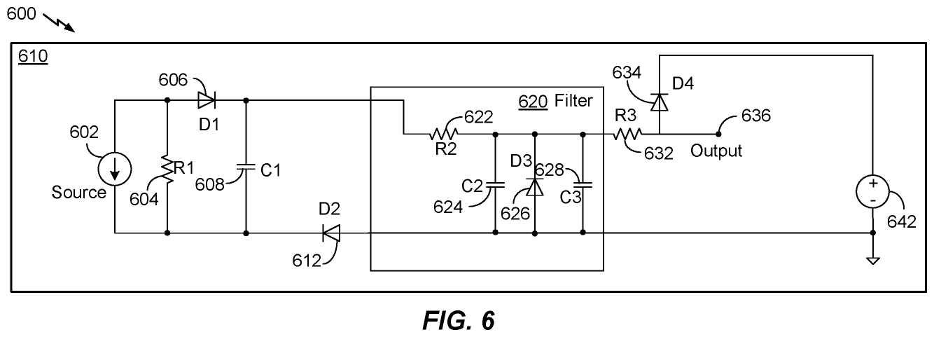

FIG. 6 is a schematic diagram showing an exemplary embodiment of a current sense circuit 600 of the AOP system of FIG. 1. The current sense circuit 600 is referred to as a “non-electrically-isolated” circuit. In an exemplary embodiment, the current sense circuit 600 comprises a current source 602 and a resistor 604. In an exemplary embodiment, the current sense circuit 600 is configured to monitor the voltage drop across the resistor 604 caused by the current flowing through, or consumed by, the current source 602. In an exemplary embodiment, the current sense circuit 600 may be an example of the first current sense element 624 or the second current sense element 626 of FIG. 4.

The example current sense circuit 600 also includes a diode 606, a capacitor 608 and a diode 612. In a non-limiting example implementation, the diode 606 and the diode 612 may be implemented using Schottky diodes.

The example current sense circuit 600 also comprises a filter 620. In an exemplary embodiment, the filter 620 comprises a resistor 622, a capacitor 624, a diode 626 and a capacitor 628. In an example implementation, the diode 626 may be a Zener (or voltage regulator) diode. The output of the filter 620 is provided to the resistor 632. In the example implementation shown in FIG. 6, the current sense circuit 600 also comprises a diode 634 and a voltage source 642. In an example implementation the diode 634 may be implemented using a Schottky diode. The output of the current sense circuit 600 may be taken over node 636.

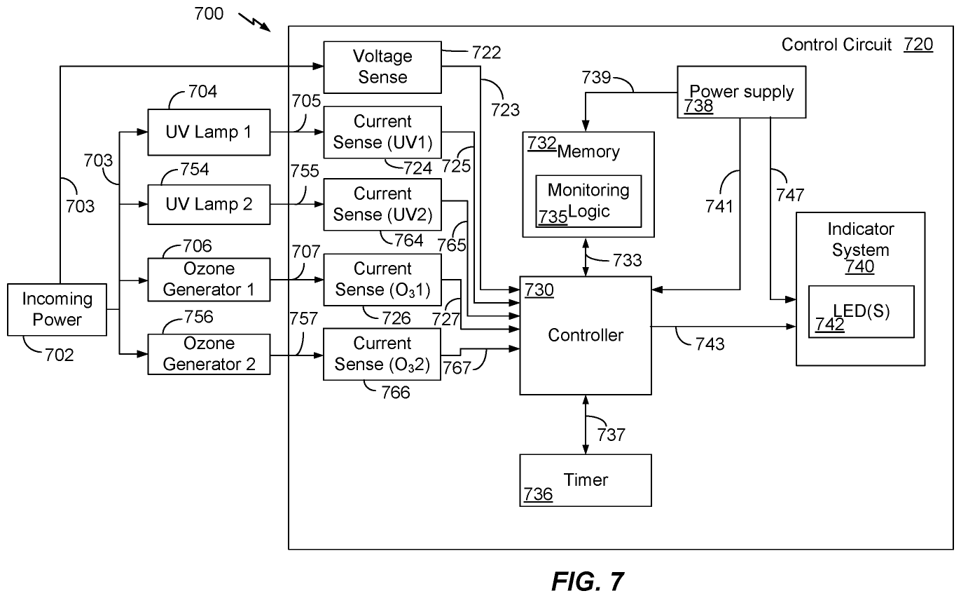

FIG. 7 is a block diagram showing an exemplary embodiment of an electrical subsystem associated with the advanced oxidation process (AOP) water treatment and sanitation system of FIG. 1. The electrical subsystem 700 shown in FIG. 7 is similar to the electrical subsystem 400 shown in FIG. 4, and as such, elements in FIG. 7 that are similar to corresponding elements in FIG. 4 will be labeled using the nomenclature 7XX, where an element in FIG. 7 labeled 7XX is similar to an element in FIG. 4 labeled 4XX.

In addition to the elements described with regard to the electrical subsystem 400 shown in FIG. 4, the electrical subsystem 700 comprises an additional UV light generating element 754 and an additional ozone generator cell 756. An additional current sense element 764 may be configured to monitor the current flowing through, or consumed by, the additional UV light generating element 754 over connection 755. Similarly, an additional current sense element 766 may be configured to monitor the current flowing through, or consumed by, the additional ozone generator cell 756 over connection 757.

The additional current sense element 764 can be configured to provide a signal indicative of the operational condition of the UV light generating element 754 to the controller 730 over connection 765; and the additional current sense element 766 can be configured to provide a signal indicative of the operational condition of the ozone generator cell 756 to the controller 730 over connection 767. In this manner, the control circuit 720 may be configured to individually monitor multiple UV light generating elements and multiple ozone generating cells.

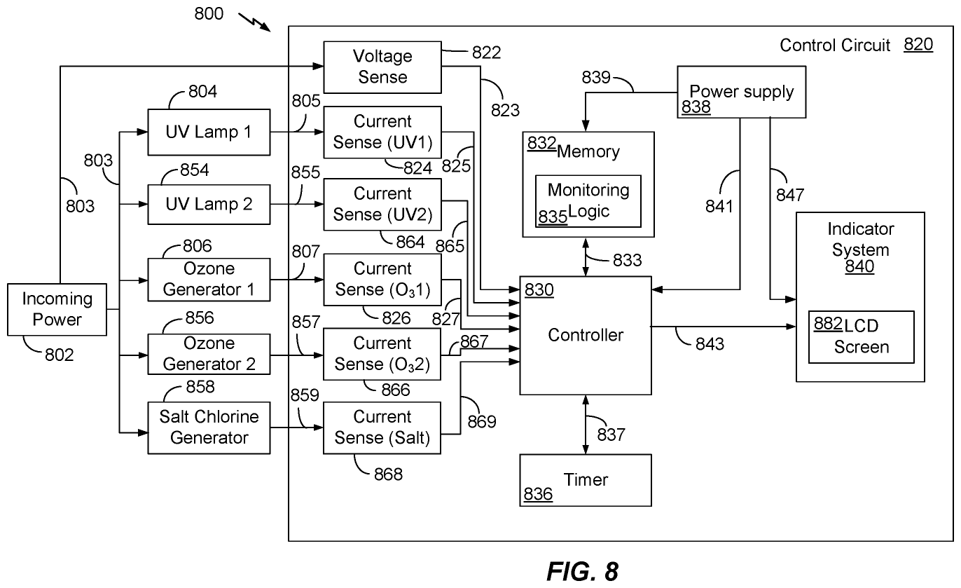

FIG. 8 is a block diagram showing an exemplary embodiment of an electrical subsystem associated with the advanced oxidation process (AOP) water treatment and sanitation system of FIG. 1. The electrical subsystem 800 shown in FIG. 8 is similar to the electrical subsystem 400 shown in FIG. 4 and electrical subsystem 700 shown in FIG. 7, and as such, elements in FIG. 8 that are similar to corresponding elements in FIG. 4 and FIG. 7 will be labeled using the nomenclature 8XX, where an element in FIG. 8 labeled 8XX is similar to an element in FIG. 4 labeled 4XX and an element in FIG. 7 labeled 7XX.

In addition to the elements described with regard to the electrical subsystem 400 shown in FIG. 4 and electrical subsystem 700 shown in FIG. 7, the electrical subsystem 800 comprises a salt chlorine generator element 858. An additional current sense element 868 may be configured to monitor the current flowing through, or consumed by, the salt chlorine generator element 858 over connection 859.

The additional current sense element 868 can be configured to provide a signal indicative of the operational condition of the salt chlorine generator element 858 to the controller 830 over connection 869. In this manner, the control circuit 820 may be configured to monitor a salt chlorine generator.

In an alternative exemplary embodiment that may be applicable to all embodiments of the control circuits of FIGS. 4, 7 and 8, the indicator system 840 in the exemplary embodiment shown in FIG. 8 also may comprise a liquid crystal display (LCD) 882, or other visual display, instead of or in addition to the LEDs mentioned herein. In such an embodiment, the indicator system 840 may be able to communicate information to a user in addition to the information communicated by the LEDs mentioned herein.

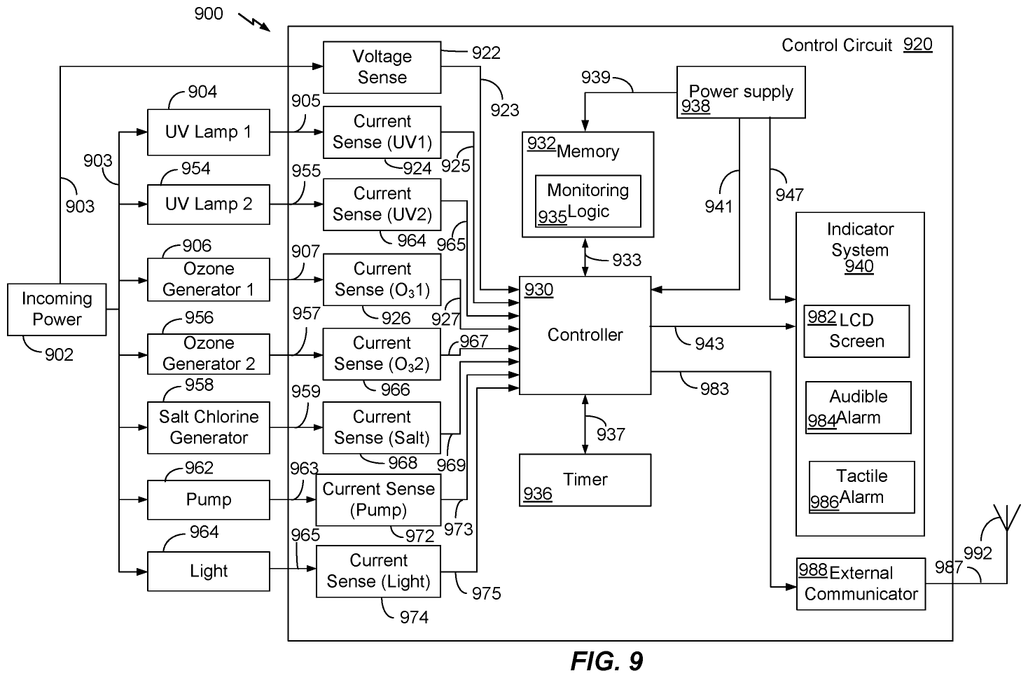

FIG. 9 is a block diagram showing an exemplary embodiment of an electrical subsystem associated with the advanced oxidation process (AOP) water treatment and sanitation system of FIG. 1. The electrical subsystem 900 shown in FIG. 9 is similar to the electrical subsystem 400 shown in FIG. 4, the electrical subsystem 700 shown in FIG. 7, and the electrical subsystem 800 shown in FIG. 8, and as such, elements in FIG. 9 that are similar to corresponding elements in FIG. 4, FIG. 7 and FIG. 8 will be labeled using the nomenclature 9XX, where an element in FIG. 9 labeled 9XX is similar to an element in FIG. 4 labeled 4XX, an element in FIG. 7 labeled 7XX, and an element in FIG. 8 labeled 8XX.

In addition to the elements described with regard to the electrical subsystem 400 shown in FIG. 4, electrical subsystem 700 shown in FIG. 7, and electrical subsystem 800 shown in FIG. 8, the electrical subsystem 900 comprises a pump 962 and a light 964. An additional current sense element 972 may be configured to monitor the current flowing through, or consumed by, the pump 962 over connection 963, and an additional current sense element 974 may be configured to monitor the current flowing through, or consumed by, the light 964 over connection 965. In an alternative exemplary embodiment, other elements, such as a mechanical chemical feeder, or another element, may be implemented.

The additional current sense element 972 can be configured to provide a signal indicative of the operational condition of the pump 962 to the controller 930 over connection 973; and the additional current sense element 974 can be configured to provide a signal indicative of the operational condition of the light 964 to the controller 930 over connection 975. In this manner, the control circuit 920 may be configured to monitor a pump, a light, or other elements.

In an alternative exemplary embodiment that may be applicable to all embodiments of the control circuits of FIGS. 4, 7, 8, and 9, the indicator system 940 in the exemplary embodiment shown in FIG. 9 also may comprise one or more of an audible alarm 984, a tactile alarm 986, or other audible or visual display or alarm, instead of or in addition to the LEDs 442 (FIG. 4) and LCD 982 mentioned herein. An audible alarm may be configured to provide a different sound for different components or different alarm conditions. A tactile alarm may be configured to provide a different vibration or different frequency of vibration for different components or different alarm conditions. In such an embodiment, the indicator system 940 may be able to communicate information to a user in addition to the information communicated by the LEDs and LCD mentioned herein.

In an exemplary embodiment, the electrical subsystem 900 may also comprise an external communication element 988 coupled to the controller 930 over connection 983. In an exemplary embodiment, the external communication element 988 may be a wired or a wireless communication device configured to provide communication access to and from the control circuit 920. If implemented as a wired communication element, the external communication element 988 may include a physical port and interface to communicate over a wired communication interface, such as, for example, a wired local area network (LAN), or a wired wide area network (WAN). If implemented as a wireless communication element, the external communication element 988 may be coupled to an antenna 992 over a connection 987 and may include circuitry to allow wireless radio frequency (RF) communication over short or long range wireless communication interfaces, such as, for example only, Bluetooth, WiFi, 3G, 4G, 5G, or other wireless communication interfaces. In an exemplary embodiment, the external communication element 988 may be configured to communicate with a cellular phone and/or a home automation system. In an exemplary embodiment, the external communication element 988 may be configured to cooperate with other communication devices or elements to, for example, automatically order replacement parts when the AOP system 100 detects that a component is approaching the end of service life, or notify the user with a reminder to order the replacement parts for upcoming maintenance. In an exemplary embodiment, the controller 930 and the memory 932 may be configured to collect and store data relating to the performance of the components that are monitored by the electrical subsystem 900, or other embodiments of the electrical subsystems described herein. For example, in an exemplary embodiment, the control circuit 920 can output data provided by the current sense circuits and collected by the controller 930 and memory 932 and provide this data as, for example, a report on each component's efficiency based on the current level collected across a given period of time (for example, if a component is running at the high end of the acceptable current range it may indicate that the components is not operating efficiently and the control circuit 920 can communicate this information to inform the user that the component is not operating at ideal efficiency). In an exemplary embodiment, this information may be communicated to a user by the eternal communicator 988 using, for example, one or more of a mobile application (an App) and may communicate using, for example, a WiFi or a Bluetooth communication link.

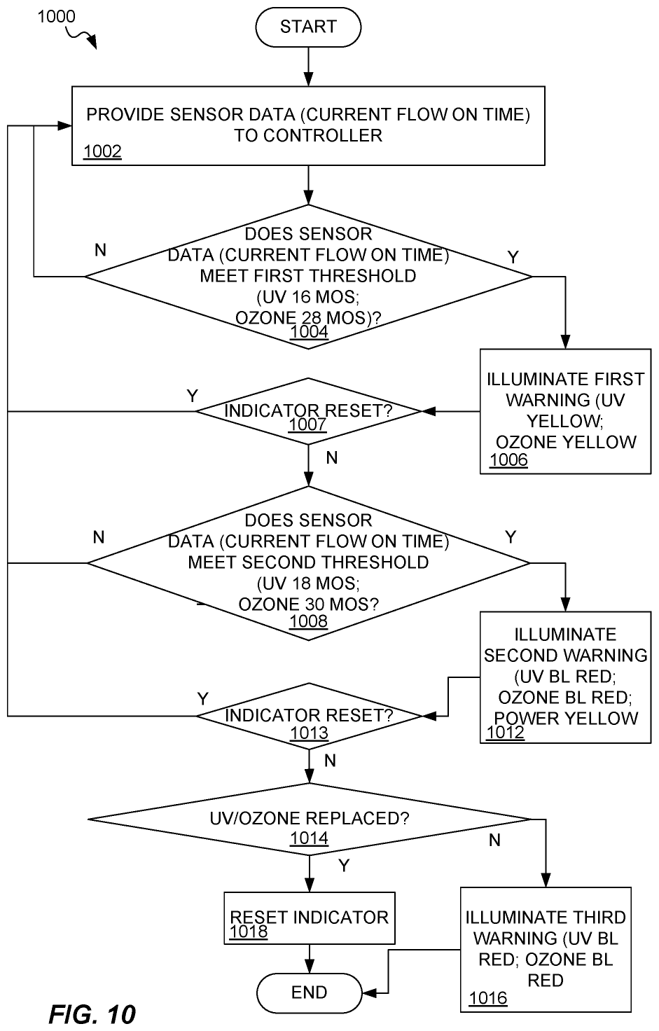

FIG. 10 is a flow chart 1000 describing an example of the operation of an AOP system. The blocks in the method 1000 can be performed in or out of the order shown, and in some embodiments, can be performed at least in part in parallel.

In block 1002, sensor data is provided to a controller. For example, a current sense element may obtain operational information related to a UV light generating element, an ozone generator cell, or another element, and provide a signal indicative of the operational information to a controller. An example of the operational information may be a total operating time of a UV light generating element, an ozone generator cell, or another element.

In block 1004 it is determined whether the sensor data meets a first threshold. For example, the sensor data relating to an operational aspect of a UV light generating element, an ozone generator cell, or another element may be compared against a first threshold. An example of a first threshold may be a preconfigured or a predetermined value relating to a maintenance time, or a service life time of a UV light generating element, an ozone generator cell, or another element. For example, a first threshold may be 16 months (or 11.5 k hours) for a UV light generating element and may be 28 months (or 20 k hours) for an ozone generator cell.

If it is determined in block 1004 that the sensor data does not meet the first threshold, then the process returns to block 1002.

If it is determined in block 1004 that the sensor data meets the first threshold, then, in block 1006, a first warning indicator may be provided. For example, if it is determined in block 1004 that a UV light generation element has been operating for 16 months (or about 11.5 k hours), then an LED (or other indicator) on the indicator system 204 may be changed from green to yellow. Similarly, for example, if it is determined in block 1004 that an ozone generator cell has been operating for 28 months (or about 20 k hours), then an LED (or other indicator) on the indicator system 204 may be changed from green to yellow.

In block 1007, it is determined whether the indicator in block 1006 has been reset. If it is determined in block 1007 that the indicator has been reset, then the process returns to block 1002. If it is determined in block 1007 that the indicator has not been reset, then the process proceeds to block 1008.

In block 1008, it is determined whether the sensor data meets a second threshold. For example, the sensor data relating to an operational aspect of a UV light generating element, an ozone generator cell, or another element may be compared against a second threshold. An example of a second threshold may be a maintenance time, or a service life time of a UV light generating element, an ozone generator cell, or another element. For example, a second threshold may be 18 months (or about 13 k hours) for a UV light generating element and may be 30 months (or about 21.5 k hours) for an ozone generator cell.

If it is determined in block 1008 that the sensor data does not meet the second threshold, then the process returns to block 1002.

If it is determined in block 1008 that the sensor data meets the second threshold, then, in block 1012, a second warning indicator may be provided. For example, if it is determined in block 1008 that a UV light generation element has been operating for 18 months (or about 13 k hours), then an LED (or other indicator) on the indicator system 204 may be changed from yellow to red. Similarly, for example, if it is determined in block 1008 that an ozone generator cell has been operating for 30 months (or about 21.5 k hours), then an LED (or other indicator) on the indicator system 204 may be changed from yellow to red.

In block 1013, it is determined whether the indicator in block 1012 has been reset. If it is determined in block 1013 that the indicator has been reset, then the process returns to block 1002. If it is determined in block 1013 that the indicator has not been reset, then the process proceeds to block 1014.

In block 1014, it is determined whether the element that caused the warning has been replaced. For example, it is determined in block 1014 whether a UV light generating element or an ozone generator cell has been replaced.

If it is determined in block 1014 that the element that caused the warning has been replaced, then the indicator can be reset in block 1018 and the process ends.

If it is determined in block 1014 that the element that caused the warning has not been replaced, then, in block 1016, a third warning indicator may be provided. For example, if it is determined in block 1014 that a UV light generation element has not been replaced after the second warning (block 1012), then an LED (or other indicator) on the indicator system 204 may be changed from red to blinking red. Similarly, for example, if it is determined in block 1014 that an ozone generator cell has not been replaced after the second warning (block 1012), then an LED (or other indicator) on the indicator system 204 may be changed from red to blinking red.

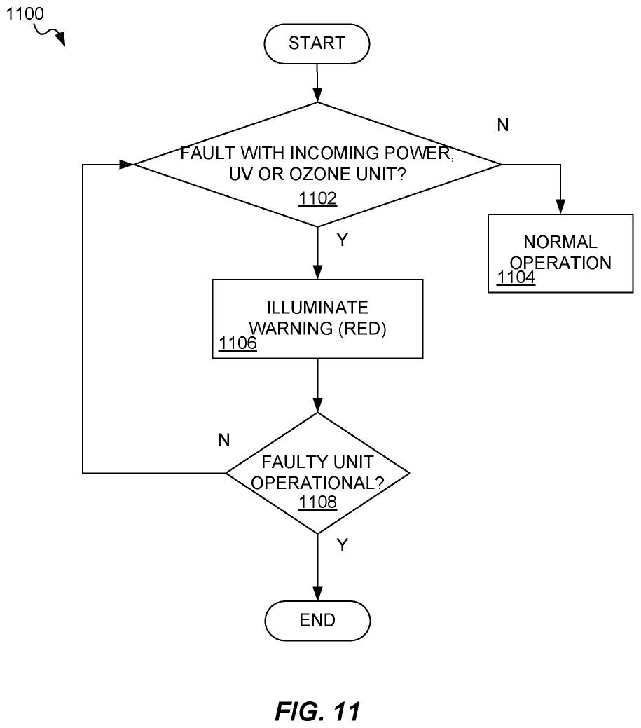

FIG. 11 is a flow chart 1100 describing an example of the operation of an AOP system. The blocks in the method 1100 can be performed in or out of the order shown, and in some embodiments, can be performed at least in part in parallel.

In block 1102, it is determined whether a fault is detected with one or more of the incoming power, UV light generating element or an ozone generator cell. An example of a fault may be an incoming voltage level that is outside of an acceptable voltage range, a connection or installation fault with a UV light generating element or an ozone generator cell, or other element, or any other anomaly that would cause an AOP system to not function correctly.

If it is determined in block 1102 that no fault is detected, then in block 1104, normal AOP operating continues.

If it is determined in block 1102 that a fault is detected, then in block 1106, an appropriate warning may be provided. For example, if an operating or installation fault is detected for incoming power, a UV light generating element, an ozone generator cell, or another element, then a corresponding LED (or other indicator) may be illuminated.

In block 1108, it is determined whether a faulty unit is operational. If it is determined in block 1108 that a faulty unit is not operational, then the process returns to block 1102.

If it is determined in block 1108 that a faulty unit is operational, then the process ends.

Although specifically described as current sense elements, other circuits, systems and methodologies may be implemented to determine the operation aspects of the UV light generating elements, ozone generator cells, and other electrical elements described herein. For example, a voltage sense element may be configured to monitor and determine the operation aspects of the UV light generating elements, ozone generator cells, and other electrical elements described herein.

It will be appreciated that the invention has been described above with reference to certain examples or preferred embodiments as shown in the drawings. Various additions, deletions, changes and alterations may be made to the above-described embodiments and examples without departing from the intended spirit and scope of this invention. Accordingly, it is intended that all such additions, deletions, changes and alterations be included within the scope of any claims in the resulting patent.

By way of example, an element, or any portion of an element, or any combination of elements may be implemented as a “processor” or a “processing system” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more exemplary embodiments, the functions described may be implemented in hardware, software, or any combination thereof. If implemented in software, the functions may be stored on or encoded as one or more instructions or code on a non-transitory computer-readable medium. Non-transitory computer-readable media include computer-readable storage media. Computer-readable storage media may be any available media that can be accessed by a computer. By way of example, and not limitation, such computer-readable storage media can comprise a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

The circuit architecture described herein may be implemented on one or more ICs, analog ICs, RFICs, mixed-signal ICs, ASICs, printed circuit boards (PCBs), electronic devices, etc. The circuit architecture described herein may also be fabricated with various IC process technologies such as complementary metal oxide semiconductor (CMOS), N-channel MOS (NMOS), P-channel MOS (PMOS), bipolar junction transistor (BJT), bipolar-CMOS (BiCMOS), silicon germanium (SiGe), gallium arsenide (GaAs), heterojunction bipolar transistors (HBTs), high electron mobility transistors (HEMTs), silicon-on-insulator (SOI), etc.

An apparatus implementing the system and circuit(s) described herein may be a stand-alone device or may be part of a larger device. A device may be (i) a stand-alone IC, (ii) a set of one or more ICs that may include memory ICs for storing data and/or instructions, (iii) an RFIC such as an RF receiver (RFR) or an RF transmitter/receiver (RTR), (iv) an ASIC such as a mobile station modem (MSM), (v) a module that may be embedded within other devices, (vi) a receiver, cellular phone, wireless device, handset, or mobile unit, (vii) etc.

As used in this description, the terms “component,” “database,” “module,” “system,” and the like are intended to refer to a computer-related entity, either hardware, firmware, a combination of hardware and software, software, or software in execution. For example, a component may be, but is not limited to being, a process running on a processor, a processor, an object, an executable, a thread of execution, a program, and/or a computer. By way of illustration, both an application running on a computing device and the computing device may be a component. One or more components may reside within a process and/or thread of execution, and a component may be localized on one computer and/or distributed between two or more computers. In addition, these components may execute from various computer readable media having various data structures stored thereon. The components may communicate by way of local and/or remote processes such as in accordance with a signal having one or more data packets (e.g., data from one component interacting with another component in a local system, distributed system, and/or across a network such as the Internet with other systems by way of the signal).

What is claimed is:

1. A system for monitoring performance of a water sanitation device comprising:
a housing having a water flow path, a power source, an ozone generating element configured to provide ozone to the water flow path, and an ultraviolet (UV) light generating element configured to expose the water in the flow path to UV light;
a first monitoring circuit configured to monitor at least one operational aspect of the ultraviolet (UV) light generating element;
a second monitoring circuit configured to monitor at least one operational aspect of the ozone generating element;
a control circuit configured to receive an output of the first monitoring circuit and an output of the second monitoring circuit; and
a display element configured to provide an indication of a status of at least one of the power source, the ultraviolet (UV) light generating element and the ozone generating element.

2. The system of claim 1, wherein the output of the first monitoring circuit comprises a first electrical current value indicative of the UV light generating element operating normally and the output of the second monitoring circuit comprises a second electrical current value indicative of the ozone generating element operating normally.

3. The system of claim 1, wherein the output of the first monitoring circuit comprises a first electrical current value indicating that the UV light generating element is properly installed and the output of the second monitoring circuit comprises a second electrical current value indicating that the ozone generating element is properly installed.

4. The system of claim 2, wherein the control circuit further comprises:
a processor coupled to a memory;
a timer coupled to the processor, the timer storing a timer value indicative of an amount of time of operation of at least one of the ozone generating element and the UV light generating element; and
a monitoring logic in the memory, the monitoring logic configured to compare the timer value to a preconfigured time value in the memory.

5. The system of claim 4, wherein the timer value indicative of an amount of time of operation of at least one of the UV light generating element and the ozone generating element corresponds to an amount of time during which at least one of the UV light generating element and the ozone generating element have operated within a predefined range of current values.

6. The system of claim 4, wherein when the timer value exceeds a first preconfigured time value in the memory, indicating on the display element that at least one of the ozone generating element and the UV light generating element is due for maintenance.

7. The system of claim 6, wherein when the timer value exceeds a second preconfigured time value in the memory, indicating on the display element that at least one of the ozone generating element and the UV light generating element is at the end of its service life.

8. The system of claim 3, wherein the control circuit further comprises:
a processor coupled to a memory; and
a monitoring logic in the memory, the monitoring logic configured to compare the first electrical current value to a first preconfigured current range and compare the second electrical current value to a second preconfigured current range, and if the first electrical current value exceeds the first preconfigured current range or the second electrical current value exceeds the second preconfigured current range, causing the display element to provide an indication that one of the UV light generating element and the ozone generating element are improperly installed.

9. The system of claim 5, further comprising:
an external communication element coupled to the processor, the external communication element configured to receive performance data relating to at least one of the UV light generating element and the ozone generating element, the external communication element configured to communicate the performance data over a wireless communication link.

10. The system of claim 1, further comprising a voltage monitoring circuit configured to monitor at least one operational aspect of the power source.

11. A method for monitoring performance of a water sanitation device, comprising:
providing sensor data relating to an operational aspect of one or more of an ozone generating element and an ultraviolet (UV) light generating element to a controller;
determining whether the sensor data indicates that a first threshold has been met;
if the first threshold has been met, causing an illumination of a first indicator signifying that the first threshold has been met;
determining whether the sensor data indicates that a second threshold has been met;
if the second threshold has been met, causing an illumination of a second indicator signifying that the second threshold has been met;
determining whether the sensor data indicates that a condition that caused the first threshold and the second threshold to be met has been removed; and
if the sensor data indicates that the condition has not been removed, causing an illumination of a third indicator signifying that the condition has not been removed.

12. The method of claim 11, wherein the sensor data relating to the operational aspect of one or more of the ozone generating element and the ultraviolet (UV) light generating element comprises an operating current.

13. The method of claim 12, further comprising monitoring an amount of time that the operating current remains within a predefined range of current values.

14. The method of claim 13, wherein determining whether the sensor data indicates that the first threshold has been met comprises:
determining the total amount of time that the operating current remains within the predefined range of current values; and
comparing the total amount of time that the operating current remains within the predefined range of current values against the first threshold.

15. The method of claim 14, wherein determining whether the sensor data indicates that the second threshold has been met comprises:
determining the total amount of time that the operating current remains within the predefined range of current values; and
comparing the total amount of time that the operating current remains within the predefined range of current values against the second threshold.

16. A method for monitoring performance of a water sanitation device, comprising:
determining whether a fault in one or more of an incoming power level, an ultraviolet (UV) light generating element and an ozone generator cell exists;
if a fault exists, causing an illumination of a first indicator signifying that the fault exists; and
if the fault is remedied, ceasing the illumination of the first indicator.

17. The method of claim 16, wherein the fault comprises one or more of an incoming voltage level that is outside of a predefined range, an installation fault with at least one of the UV light generating element and the ozone generator cell.
